SEQUENCE LISTING (32 sequences)

SEQ ID NO: 1 - protein, 469 aa, unidentified
MAMLYGKHNHEAEEYLEPVFGAPREQHDLPKYRLPKHSLSPREADRLVRDELLDEGNSRLNLATFCQTYMETEAVELMKDTLRKNAIRKSEYPRTAEIENRCVNIIANLWHAPDDEHFTGTSTTGSSEACMLGGLAMKFAWRKRAQAAGLDLNTHRPNLVISAGYQVCWEKFCVYWDVDMHVVPVDEQHMVLDVNHVLDYVDEYIIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSKLGGELPTMAINFSHSAAQLIGQYYNFIRFGMDGYREIQTKTHDVARYLAAALDKVGEFKMINNGHQLPLICYQLAPREDREWTLYDLSDRLLMNGWQVPTYPFPANLEQQVIQRIVVRADFGMNMAHDFMDDLTKAVHDLNHAHIVYHHDAAPKKYLFTH

SEQ ID NO: 2 - DNA, 1410 bp, synthetic construct
  source 1..1410: mol_type = other DNA
atggcaatgttatacggtaaacacaatcatgaagctgaagaatacttggaaccagtctttggtgcgcctcgggaacaacatgatcttcctaagtatcggttaccaaagcattcattatcccctcgagaagccgatcgcttagttcgtgatgaattattagatgaaggcaattcacgactgaacctggcaactttttgtcagacctatatggaaaccgaagccgttgaattgatgaaggatacgctgcgaaagaatgccatccggaaatctgagtacccccgcacggccgagattgaaaatcggtgtgtgaacattattgccaatctgtggcacgcacctgatgacgaacactttacgggtacctctacgacaggctcctctgaagcttgtatgttaggcggtttagcaatgaaattcgcctggcgtaaacgcgctcaagcggcaggtttagatctgaatacccatcgacctaacctcgttatttcggctggctatcaagtttgctgggaaaagttttgtgtctactgggacgttgacatgcacgtggtcccagtggatgagcaacacatggtccttgacgttaaccacgtcttagactacgtggacgaatacattattggtatcgtcggtatcatgggcatcacttataccggtcaatatgacgacctagccgcactcgataaggtcgttactcactacaatcatcagcatcccaaattaccagtctacattcacgttgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagtaaattaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagccccgcgcgaagatcgtgaatggaccctttatgatttatcggatcgcctattaatgaacggttggcaagtaccaacgtatcctttccctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacctattcacacactga

SEQ ID NO: 3 - protein, 469 aa, unidentified
MAMLYGKHNHEAEEYLEPVFGAPSEQHDLPKYRLPKHSLSPREADRLVRDELLDEGNSRLNLATFCQTYMETEAVELMKDTLRKNAIDKSEYPRTAEIENRCVNIIANLWHAPDDEHFTGTSTTGSSEACMLGGLAMKFAWRKRAQAAGLDLNTHRPNLVISAGYQVCWEKFCVYWDVDMHVVPVDEQHMVLDVNHVLDYVDEYIIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSYLGGELPTMAINFSHSAAQLIGQYYNFIRFGMDGYREIQTKTHDVARYLAAALDKVGEFKMINNGHQLPLICYQLAPREDREWTLYDLSDRLLMNGWQVPTYPFPANLEQQVIQRIVVRADFGMNMAHDFMDDLTKAVHDLNHAHIVYHHDAAPKKYLFTH

SEQ ID NO: 4 - DNA, 1410 bp, synthetic construct
  source 1..1410: mol_type = other DNA
atggcaatgttatacggtaaacacaatcatgaagctgaagaatacttggaaccagtctttggtgcgccttctgaacaacatgatcttcctaagtatcggttaccaaagcattcattatcccctcgagaagccgatcgcttagttcgtgatgaattattagatgaaggcaattcacgactgaacctggcaactttttgtcagacctatatggaaaccgaagccgttgaattgatgaaggatacgctgcgaaagaatgccatcgacaaatctgagtacccccgcacggccgagattgaaaatcggtgtgtgaacattattgccaatctgtggcacgcacctgatgacgaacactttacgggtacctctacgacaggctcctctgaagcttgtatgttaggcggtttagcaatgaaattcgcctggcgtaaacgcgctcaagcggcaggtttagatctgaatacccatcgacctaacctcgttatttcggctggctatcaagtttgctgggaaaagttttgtgtctactgggacgttgacatgcacgtggtcccagtggatgagcaacacatggtccttgacgttaaccacgtcttagactacgtggacgaatacattattggtatcgtcggtatcatgggcatcacttataccggtcaatatgacgacctagccgcactcgataaggtcgttactcactacaatcatcagcatcccaaattaccagtctacattcacgttgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagttatttaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagccccgcgcgaagatcgtgaatggaccctttatgatttatcggatcgcctattaatgaacggttggcaagtaccaacgtatcctttccctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacctattcacacactga

SEQ ID NO: 5 - protein, 469 aa, unidentified
MAMLYGKHNHEAEEYLEPVFGAPSEQHDLPKYRLPKHSLSPREADRLVRDELLDEGNSRLNLATFCQTYMEPEAVELMKDTLRKNAIDKSEYPRTAEIENRCVNIIANLWHAPDDEHFTGTSTTGSSEACMLGGLAMKFAWRKRAQAAGLDLNAHRPNLVISAGYQVCWEKFCVYWDVDMHVVPMDEQHMVLDVNHVLDYVDEYIIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSYLGGELPTMAINFSHSAAQLIGQYYNFIRFGMDGYREIQTKTHDVARYLAAALDKVGEFKMINNGHQLPLICYQLAPREDREWTLYDLSDRLLMNGWQVPTYPLPANLEQQVIQRIVVRADFGMNMAHDFMDDLTKAVHDLNHAHIVYHHDAAPKKYLFTH

SEQ ID NO: 6 - DNA, 1410 bp, synthetic construct
  source 1..1410: mol_type = other DNA
atggcaatgttatacggtaaacacaatcatgaagctgaagaatacttggaaccagtctttggtgcgccttctgaacaacatgatcttcctaagtatcggttaccaaagcattcattatcccctcgagaagccgatcgcttagttcgtgatgaattattagatgaaggcaattcacgactgaacctggcaactttttgtcagacctatatggaacccgaagccgttgaattgatgaaggatacgctgcgaaagaatgccatcgacaaatctgagtacccccgcacggccgagattgaaaatcggtgtgtgaacattattgccaatctgtggcacgcacctgatgacgaacactttacgggtacctctacgacaggctcctctgaagcttgtatgttaggcggtttagcaatgaaattcgcctggcgtaaacgcgctcaagcggcaggtttagatctgaatgcccatcgacctaacctcgttatttcggctggctatcaagtttgctgggaaaagttttgtgtctactgggacgttgacatgcacgtggtcccaatggatgagcaacacatggtccttgacgttaaccacgtcttagactacgtggacgaatacattattggtatcgtcggtatcatgggcatcacttataccggtcaatatgacgacctagccgcactcgataaggtcgttactcactacaatcatcagcatcccaaattaccagtctacattcacgttgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagttatttaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagccccgcgcgaagatcgtgaatggaccctttatgatttatcggatcgcctattaatgaacggttggcaagtaccaacgtatcctttacctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacctattcacacactga

SEQ ID NO: 7 - protein, 469 aa, unidentified
MAMLYGKHNHEAEEYLEPVFGAPREQHDLPKYRLPKHSLSPREADRLVRDELLDEGNSRLNLATFCQTYMETEAVELMKDTLRKNAIDKSEYPRTAEIENRCVNIIANLWHAPDDEHFTGTSTTGSSEACMLGGLAMKFAWRKRAQAAGLDLNTHRPNLVISAGYQVCWEKFCVYWDVDMHVVPVDEQHMVLDVNHVLDYVDEYIIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSYLGGELPTMAINFSHSAAQLIGQYYNFIRFGMDGYREIQTKTHDVARYLAAALDKVGEFKMINNGHQLPLICYQLAPREDREWTLYDLSDRLLMNGWQVPTYPFPANLEQQVIQRIVVRADFGMNMAHDFMDDLTKAVHDLNHAHIVYHHDAAPKKYLFTH

SEQ ID NO: 8 - DNA, 1410 bp, synthetic construct
  source 1..1410: mol_type = other DNA
atggcaatgttatacggtaaacacaatcatgaagctgaagaatacttggaaccagtctttggtgcgcctcgcgaacaacatgatcttcctaagtatcggttaccaaagcattcattatcccctcgagaagccgatcgcttagttcgtgatgaattattagatgaaggcaattcacgactgaacctggcaactttttgtcagacctatatggaaaccgaagccgttgaattgatgaaggatacgctgcgaaagaatgccatcgacaaatctgagtacccccgcacggccgagattgaaaatcggtgtgtgaacattattgccaatctgtggcacgcacctgatgacgaacactttacgggtacctctacgacaggctcctctgaagcttgtatgttaggcggtttagcaatgaaattcgcctggcgtaaacgcgctcaagcggcaggtttagatctgaatacccatcgacctaacctcgttatttcggctggctatcaagtttgctgggaaaagttttgtgtctactgggacgttgacatgcacgtggtcccagtggatgagcaacacatggtccttgacgttaaccacgtcttagactacgtggacgaatacattattggtatcgtcggtatcatgggcatcacttataccggtcaatatgacgacctagccgcactcgataaggtcgttactcactacaatcatcagcatcccaaattaccagtctacattcacgttgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagttatttaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagccccgcgcgaagatcgtgaatggaccctttatgatttatcggatcgcctattaatgaacggttggcaagtaccaacgtatcctttccctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacctattcacacactga

SEQ ID NO: 9 - protein, 469 aa, unidentified
MAMLYGKHNHEAEEYLEPVFGAPREQHDLPKYRLPKHSLSPREADRLVRDELLDEGNSRLNLATFCQTYMETEAVELMKDTLRKNAIRKSEYPRTAEIENRCVNIIANLWHAPDDEHFTGTSTTGSSEACMLGGLAMKFAWRKRAQAAGLDLNTHRPNLVISAGYQVCWEKFCVYWDVDMHVVPVDEQHMVLDVNHVLDYVDEYIIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSYLGGELPTMAINFSHSAAQLIGQYYNFIRFGMDGYREIQTKTHDVARYLAAALDKVGEFKMINNGHQLPLICYQLAPREDREWTLYDLSDRLLMNGWQVPTYPFPANLEQQVIQRIVVRADFGMNMAHDFMDDLTKAVHDLNHAHIVYHHDAAPKKYLFTH

SEQ ID NO: 10 - DNA, 1410 bp, synthetic construct
  source 1..1410: mol_type = other DNA
atggcaatgttatacggtaaacacaatcatgaagctgaagaatacttggaaccagtctttggtgcgcctcgcgaacaacatgatcttcctaagtatcggttaccaaagcattcattatcccctcgagaagccgatcgcttagttcgtgatgaattattagatgaaggcaattcacgactgaacctggcaactttttgtcagacctatatggaaaccgaagccgttgaattgatgaaggatacgctgcgaaagaatgccatccggaaatctgagtacccccgcacggccgagattgaaaatcggtgtgtgaacattattgccaatctgtggcacgcacctgatgacgaacactttacgggtacctctacgacaggctcctctgaagcttgtatgttaggcggtttagcaatgaaattcgcctggcgtaaacgcgctcaagcggcaggtttagatctgaatacccatcgacctaacctcgttatttcggctggctatcaagtttgctgggaaaagttttgtgtctactgggacgttgacatgcacgtggtcccagtggatgagcaacacatggtccttgacgttaaccacgtcttagactacgtggacgaatacattattggtatcgtcggtatcatgggcatcacttataccggtcaatatgacgacctagccgcactcgataaggtcgttactcactacaatcatcagcatcccaaattaccagtctacattcacgttgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagttatttaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagccccgcgcgaagatcgtgaatggaccctttatgatttatcggatcgcctattaatgaacggttggcaagtaccaacgtatcctttccctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacctattcacacactga

SEQ ID NO: 11 - protein, 469 aa, unidentified
MAMLYGKHNHEAEEYLEPVFGAPREQHDLPKYRLPKHSLSPREADRLVRDELLDEGNSRLNLATFCQTYMETEAVELMKDTLRKNAIDKSEYPRTAEIENRCVNIIANLWHAPDDEHFTGTSTTGSSEACMLGGLAMKFAWRKRAQAAGLDLNTHRPNLVISAGYQVCWEKFCVYWDVDMHVVPVDEQHMVLDVNHVLDYVDEYIIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSKLGGELPTMAINFSHSAAQLIGQYYNFIRFGMDGYREIQTKTHDVARYLAAALDKVGEFKMINNGHQLPLICYQLAPREDREWTLYDLSDRLLMNGWQVPTYPFPANLEQQVIQRIVVRADFGMNMAHDFMDDLTKAVHDLNHAHIVYHHDAAPKKYLFTH

SEQ ID NO: 12 - DNA, 1410 bp, synthetic construct
  source 1..1410: mol_type = other DNA
atggcaatgttatacggtaaacacaatcatgaagctgaagaatacttggaaccagtctttggtgcgcctcgcgaacaacatgatcttcctaagtatcggttaccaaagcattcattatcccctcgagaagccgatcgcttagttcgtgatgaattattagatgaaggcaattcacgactgaacctggcaactttttgtcagacctatatggaaaccgaagccgttgaattgatgaaggatacgctgcgaaagaatgccatcgacaaatctgagtacccccgcacggccgagattgaaaatcggtgtgtgaacattattgccaatctgtggcacgcacctgatgacgaacactttacgggtacctctacgacaggctcctctgaagcttgtatgttaggcggtttagcaatgaaattcgcctggcgtaaacgcgctcaagcggcaggtttagatctgaatacccatcgacctaacctcgttatttcggctggctatcaagtttgctgggaaaagttttgtgtctactgggacgttgacatgcacgtggtcccagtggatgagcaacacatggtccttgacgttaaccacgtcttagactacgtggacgaatacattattggtatcgtcggtatcatgggcatcacttataccggtcaatatgacgacctagccgcactcgataaggtcgttactcactacaatcatcagcatcccaaattaccagtctacattcacgttgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagtaaattaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagccccgcgcgaagatcgtgaatggaccctttatgatttatcggatcgcctattaatgaacggttggcaagtaccaacgtatcctttccctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacctattcacacactga

SEQ ID NO: 13 - DNA, 27 bp, synthetic construct
  source 1..27: mol_type = other DNA
atggcaatgttatacggtaaacacaat

SEQ ID NO: 14 - DNA, 27 bp, synthetic construct
  source 1..27: mol_type = other DNA
cttaggaagatcatgttgttcgcgagg

SEQ ID NO: 15 - DNA, 27 bp, synthetic construct
  source 1..27: mol_type = other DNA
ggtgcgcctcgcgaacaacatgatctt

SEQ ID NO: 16 - DNA, 27 bp, synthetic construct
  source 1..27: mol_type = other DNA
tcagatttccggatggcattctttcgc

SEQ ID NO: 17 - DNA, 27 bp, synthetic construct
  source 1..27: mol_type = other DNA
tgccatccggaaatctgagtacccccg

SEQ ID NO: 18 - DNA, 27 bp, synthetic construct
  source 1..27: mol_type = other DNA
cattgctttaccgcctaacatacaagc

SEQ ID NO: 19 - DNA, 27 bp, synthetic construct
  source 1..27: mol_type = other DNA
aggcggtaaagcaatgaaattcgcctg

SEQ ID NO: 20 - DNA, 27 bp, synthetic construct
  source 1..27: mol_type = other DNA
aaacttacgccagcaaacttgatagcc

SEQ ID NO: 21 - DNA, 27 bp, synthetic construct
  source 1..27: mol_type = other DNA
ttgctggcgtaagttttgtgtctactg

SEQ ID NO: 22 - DNA, 27 bp, synthetic construct
  source 1..27: mol_type = other DNA
taagacacggttaacgtcaaggaccat

SEQ ID NO: 23 - DNA, 27 bp, synthetic construct
  source 1..27: mol_type = other DNA
gttaaccgtgtcttagactacgtggac

SEQ ID NO: 24 - DNA, 27 bp, synthetic construct
  source 1..27: mol_type = other DNA
atcgagtttggctaggtcgtcatattg

SEQ ID NO: 25 - DNA, 27 bp, synthetic construct
  source 1..27: mol_type = other DNA
ctagccaaactcgataaggtcgttact

SEQ ID NO: 26 - DNA, 27 bp, synthetic construct
  source 1..27: mol_type = other DNA
cccacctaatttactaactttgaagac

SEQ ID NO: 27 - DNA, 27 bp, synthetic construct
  source 1..27: mol_type = other DNA
aaagttagtaaattaggtggggagttg

SEQ ID NO: 28 - DNA, 27 bp, synthetic construct
  source 1..27: mol_type = other DNA
cagagcgcgtgccaggtagcgggcaac

SEQ ID NO: 29 - DNA, 27 bp, synthetic construct
  source 1..27: mol_type = other DNA
ctggcacgcgctctggataaagttggt

SEQ ID NO: 30 - DNA, 27 bp, synthetic construct
  source 1..27: mol_type = other DNA
ttgttgtttcagattagcagggaaagg

SEQ ID NO: 31 - DNA, 27 bp, synthetic construct
  source 1..27: mol_type = other DNA
aatctgaaacaacaagtcatccaacga

SEQ ID NO: 32 - DNA, 27 bp, synthetic construct
  source 1..27: mol_type = other DNA
tcagtgtgtgaataggtatttcttagg

(19) United States
(12) Patent Application Publication
(10) Pub. No.: US 2024/0110173 A1
(43) Pub. Date: Apr. 4, 2024

(54) GLUTAMATE DECARBOXYLASE MUTANT WITH IMPROVED PH TOLERANCE AND USE THEREOF IN SYNTHESIS OF GAMMA-AMINOBUTYRIC ACID

(71) Applicant: Jiangnan University, Wuxi (CN)

(72) Inventors: Zhiming RAO, Wuxi (CN); Jin Han, Wuxi (CN); Taowei Yang, Wuxi (CN); Meijuan Xu, Wuxi (CN); Xian Zhang, Wuxi (CN)

(21) Appl. No.: 18/542,109

(22) Filed: Dec. 15, 2023

Related U.S. Application Data
(63) Continuation of application No. PCT/CN22/76360, filed on Feb. 15, 2022.

(30) Foreign Application Priority Data
Jan. 25, 2022 (CN) 202210086048.3

(51) Int. Cl. C12N 9/88; C12P 13/00

(52) U.S. Cl. CPC C12N 9/88; C12P 13/005; C12Y 401/01015

(57) ABSTRACT

The invention provides a glutamate decarboxylase mutant with improved pH tolerance and use thereof in synthesis of gamma-aminobutyric acid. The mutant is obtained by mutating glutamate decarboxylase having an amino acid sequence as shown in SEQ ID NO. 3. The enzyme activity of the mutant at pH 6.5 is improved to 178% of the original enzyme (SEQ ID NO. 3). The final yield of 1000 g of substrate fed in batches in a 5L tank for 12 h is up to 688.13 g/L, which is about 52% higher than the productivity of the original glutamate decarboxylase. The final molar conversion rate can reach 98.2%. The invention not only broadens the enzyme activity of GAD under the optimum pH, but also broadens the enzyme activity of GAD under the neutral pH, and enhances the capability of the GAD to synthesize gamma-aminobutyric acid, and therefore is more suitable for industrial production.

This application is a Continuation Application of PCT/CN2022/076360, filed on Feb. 15, 2022, which claims priority to Chinese Patent Application No. 202210086048.3, filed on Jan. 25, 2022, which is incorporated by reference for all purposes as if fully set forth herein.

A Sequence Listing XML file named “10015_0140.xml” created on Dec. 15, 2023, and having a size of 39,710 bytes, is filed concurrently with the specification. The sequence listing contained in the XML file is part of the specification and is herein incorporated by reference in its entirety.

### FIELD OF THE INVENTION

The invention belongs to the technical field of bioengineering, in particular to a glutamate decarboxylase mutant with improved pH tolerance and use thereof in synthesis of gamma-aminobutyric acid.

### DESCRIPTION OF THE RELATED ART

GABA (gamma-aminobutyric acid) is a natural non-protein amino acid. As the main inhibitory neurotransmitter in the central nervous system of mammals, GABA has many important physiological functions such as anti-anxiety, lowering blood pressure, calming nerves, enhancing memory, preventing obesity and diabetes, etc., and therefore, has been widely used in medicine and food fields.

At present, the methods for preparing GABA are mainly divided into two types: chemical synthesis and biocatalysis. According to the different types of biocatalysts, biocatalysis methods may be further divided into a plant transformation method, a microbial transformation method, and an enzymatic method. There are mainly two chemical synthesis methods commonly used for preparing GABA. In one method, potassium phthalimide and γ-chlorobutyl cyanide are used as substrates and reacted at 180° C., and the product is hydrolyzed with concentrated sulfuric acid in reflux and then crystallized and purified to obtain GABA. In another method, pyrrolidone as a raw material is reacted at 121° C.-135° C., followed by a ring-opening hydrolysis with Ca(OH)₂ and NH₄HCO₃ to prepare GABA. Because of the harsh reaction conditions in industrial production and the easy production of toxic substances in the reaction process, the production of GABA is mainly by microbial enzymatic synthesis. Glutamate decarboxylase (GAD) can catalyze the decarboxylation of L-glutamic acid to obtain gamma-aminobutyric acid, and one proton is consumed in the reaction process.

GAD is widely present in bacteria, yeast and filamentous fungi, and mediates intracellular GABA synthesis. The preparation of GABA through transformation of microorganisms containing GAD is not limited by resources, environment and space, has become one of the most effective means for industrial production of GABA, and has attracted widespread attention. At present, the optimum pH of GAD in nature is generally 4.0 to 5.0, and the enzyme activity drops sharply at neutral pH. In the process of natural fermentation or transformation of microorganisms, the catalytic activity of bacteria often decreases with the increase of pH value caused by GABA accumulation in the fermentation broth, resulting in a low substrate conversion rate. Therefore, broadening the pH range of glutamate decarboxylase can help improve the substrate conversion rate.

### SUMMARY OF THE INVENTION

An object of the invention is to provide a glutamate decarboxylase mutant with improved pH tolerance and use thereof in synthesis of gamma-aminobutyric acid, where the enzyme activity is improved under partial neutral conditions, and the conversion efficiency for preparing gamma-aminobutyric acid can be improved.

According to the technical scheme of the invention, the glutamate decarboxylase mutant with improved pH tolerance is obtained by mutating glutamate decarboxylase having an amino acid sequence as shown in SEQ ID NO. 3 in any of the following ways:
(1) mutating serine at position 24 to arginine, with an amino acid sequence as shown in SEQ ID NO. 7;
(2) mutating serine at position 24 to arginine, and mutating aspartic acid at position 88 to arginine, with an amino acid sequence as shown in SEQ ID NO. 9;
(3) mutating serine at position 24 to arginine, and mutating tyrosine at position 309 to lysine, with an amino acid sequence as shown in SEQ ID NO. 11; and
(4) mutating serine at position 24 to arginine, mutating aspartic acid at position 88 to arginine, and mutating tyrosine at position 309 to lysine, with an amino acid sequence as shown in SEQ ID NO. 1.

Compared with GB 01-21 strain glutamate decarboxylase (with an amino acid sequence as shown in SEQ ID NO. 5, and a nucleotide sequence as shown in SEQ ID NO. 6), the glutamate decarboxylase as shown in SEQ ID NO. 3 has four amino acid residue mutations, namely, mutation of proline at position 72 to threonine, mutation of alanine at position 154 to threonine, mutation of methionine at position 185 to valine, and mutation of leucine at position 412 to phenylalanine.

A nucleotide sequence of a gene encoding the glutamate decarboxylase as shown in SEQ ID NO. 3 is as shown in SEQ ID NO. 4.

A second aspect of the invention provides a gene encoding the glutamate decarboxylase mutant with improved pH tolerance.

Preferably, a nucleotide sequence of the gene is as shown in SEQ ID NO. 2, SEQ ID NO. 8, SEQ ID NO. 10, or SEQ ID NO. 12.

Specifically, a nucleotide sequence of a gene encoding the glutamate decarboxylase mutant having an amino acid sequence as shown in SEQ ID NO. 1 is as shown in SEQ ID NO. 2. A nucleotide sequence of a gene encoding the glutamate decarboxylase mutant having an amino acid sequence as shown in SEQ ID NO. 7 is as shown in SEQ ID NO. 8. A nucleotide sequence of a gene encoding the glutamate decarboxylase mutant having an amino acid sequence as shown in SEQ ID NO. 9 is as shown in SEQ ID NO. 10. A nucleotide sequence of a gene encoding the glutamate decarboxylase mutant having an amino acid sequence as shown in SEQ ID NO. 11 is as shown in SEQ ID NO. 12.

A third aspect of the invention provides a recombinant expression vector carrying the gene.

Preferably, pET-28a, PMA5, or PXMJ-19 is used as an original expression vector.

A fourth aspect of the invention provides a recombinant strain including the recombinant expression vector.

Preferably, Escherichia Coli, Bacillus subtilis, or Corynebacterium glutamicum is used as a host strain of the recombinant strain.

Preferably, the Bacillus subtilis includes Bacillus subtilis 168 (BS168).

A fifth aspect of the invention provides use of the glutamate decarboxylase mutant with improved pH tolerance, the gene, the recombinant expression vector, or the recombinant strain in synthesis of gamma-aminobutyric acid.

Preferably, gamma-aminobutyric acid is produced by using L-glutamic acid or L-sodium glutamate as a substrate and the glutamate decarboxylase mutant with improved pH tolerance or the recombinant strain as a catalyst.

Preferably, a 0.9% NaCl solution is used as a conversion system, no pH adjustment is required, and the product is easily extracted after conversion.

Preferably, the temperature is 25° C.-60° C.

The technical solution of the invention has the following advantages compared to the prior art:

By mutating glutamate decarboxylase, the invention not only broadens the enzyme activity of GAD under the optimum pH, but also broadens the enzyme activity of GAD under the neutral pH, and enhances the capability of the GAD to synthesize gamma-aminobutyric acid, and therefore is more suitable for industrial production.

Test results show that the enzyme activity of the glutamate decarboxylase mutant of the invention increased under different pH conditions compared with the parental glutamate decarboxylase, especially when the mutation mode was that serine at position 24 was mutated to arginine, aspartate at position 88 was mutated to arginine, and tyrosine residue at position 309 was mutated to lysine residue. The enzyme activity of the mutant at pH 6.5 is improved to 178% of the original enzyme (as shown in SEQ ID NO. 3). The final yield of 1000 g of substrate fed in batches in a 5 L tank for 12 h is up to 688.13 g/L, which is about 52% higher than the productivity of the original glutamate decarboxylase. The final molar conversion rate can reach 98.2%.

### BRIEF DESCRIPTION OF THE DRAWINGS

FIG. 1 is an SDS-PAGE diagram of a mutant strain.

FIG. 2 shows relative enzyme activities of different mutants.

FIG. 3 shows relative enzyme activities of a mutant strain with three combined mutations at different pH.

FIG. 4 shows relative enzyme activities of a mutant strain with three combined mutations at different temperatures.

### DETAILED DESCRIPTION OF THE PREFERRED EMBODIMENTS

The invention will be further described below in conjunction with the accompanying drawings and specific embodiments, so that those skilled in the art can better understand and implement the invention, but the embodiments described are not intended to limit the invention.

### Example 1 Construction of a Recombinant Expression Vector Including a Gene Encoding a Glutamate Decarboxylase Mutant

Mutant genes were constructed based on a fusion PCR method by using a recombinant plasmid pET-28a including a nucleotide sequence as shown in SEQ ID NO. 4 as a template and respectively using primers containing mutation sites to amplify gene fragments upstream and downstream the mutation sites. For genes with combined mutations, by using a single-point mutant plasmid as a template and using primers containing mutation sites, gene fragments upstream and downstream the mutation sites were amplified, and then a series of genes can be obtained by using the fusion PCR method. Details are given as follows.

| Primer | Gene |
|---|---|
| P1/P2, P3/P20 | lpgad^{S24R} |
| P1/P4, P5/P20 | lpgad^{D88R} |
| P1/P6, P7/P20 | Lpgad^{L135K} |
| P1/P8, P9/P20 | Lpgad^{E170R} |
| P1/P10, P11/P20 | Lpgad^{H196R} |
| P1/P12, P13/P20 | Lpgad^{A225K} |
| P1/P14, P15/P20 | Lpgad^{Y309K} |
| P1/P16, P17/P20 | Lpgad^{A359R} |
| P1/P18, P19/P20 | Lpgad^{E417K} |
| P1/P2, P3/P4, P5/P20 | lpgad^{S24R/D88R} |
| P1/P2, P3/P6, P7/P20 | lpgad^{S24R/L135K} |
| P1/P2, P3/P8, P9/P20 | lpgad^{S24R/E170R} |
| P1/P2, P3/P10, P11/P20 | lpgad^{S24R/H196R} |
| P1/P2, P3/P12, P13/P20 | lpgad^{S24R/A225K} |
| P1/P2, P3/P14, P15/P20 | lpgad^{S24R/Y309K} |
| P1/P2, P3/P16, P17/P20 | lpgad^{S24R/A359R} |
| P1/P2, P3/P18, P19/P20 | lpgad^{S24R/E417K} |
| P1/P2, P3/P4, P5/P14, P15/P20 | lpgad^{S24R/D88R/Y309K} |

| | | |
|---|---|---|
| P1 | (SEQ ID NO. 13): | ATGGCAATGTTATACGGTAAACACAAT |
| P2 | (SEQ ID NO. 14): | CTTAGGAAGATCATGTTGTTCGCGAGG |
| P3 | (SEQ ID NO. 15): | GGTGCGCCTCGCGAACAACATGATCTT |
| P4 | (SEQ ID NO. 16): | TCAGATTTCCGGATGGCATTCTTTCGC |
| P5 | (SEQ ID NO. 17): | TGCCATCCGGAAATCTGAGTACCCCCG |
| P6 | (SEQ ID NO. 18): | CATTGCTTTACCGCCTAACATACAAGC |
| P7 | (SEQ ID NO. 19): | AGGCGGTAAAGCAATGAAATTCGCCTG |
| P8 | (SEQ ID NO. 20): | AAACTTACGCCAGCAAACTTGATAGCC |
| P9 | (SEQ ID NO. 21): | TTGCTGGCGTAAGTTTTGTGTCTACTG |
| P10 | (SEQ ID NO. 22): | TAAGACACGGTTAACGTCAAGGACCAT |
| P11 | (SEQ ID NO. 23): | GTTAACCGTGTCTTAGACTACGTGGAC |
| P12 | (SEQ ID NO. 24): | ATCGAGTTTGGCTAGGTCGTCATATTG |
| P13 | (SEQ ID NO. 25): | CTAGCCAAACTCGATAAGGTCGTTACT |
| P14 | (SEQ ID NO. 26): | CCCACCTAATTTACTAACTTTGAAGAC |
| P15 | (SEQ ID NO. 27): | AAAGTTAGTAAATTAGGTGGGGAGTTG |
| P16 | (SEQ ID NO. 28): | CAGAGCGCGTGCCAGGTAGCGGGCAAC |
| P17 | (SEQ ID NO. 29): | CTGGCACGCGCTCTGGATAAAGTTGGT |
| P18 | (SEQ ID NO. 30): | TTGTTGTTTCAGATTAGCAGGGAAAGG |
| P19 | (SEQ ID NO. 31): | AATCTGAAACAACAAGTCATCCAACGA |
| P20 | (SEQ ID NO. 32): | TCAGTGTGTGAATAGGTATTTCTTAGG |

The expression plasmid pET-28a was enzymatically cleaved by the restriction endonuclease EcoR I/Hind III to obtain a linearized vector. A gene of interest was ligated to the linearized vector to construct a recombinant plasmid. The recombinant plasmid was transformed into Escherichia coli JM109 by chemical transformation, coated on an LB plate containing kanamycin, and incubated overnight at 37° C. Clones were randomly picked up, identified by colony PCR and verified by sequencing.

Results show that the recombinant expression vector containing the gene encoding the glutamate decarboxylase mutant was successfully transformed into the cloning vector Escherichia coli JM109, and the recombinant plasmid was extracted from the bacterial solution with successful mutation as verified by sequencing, and stored in a refrigerator at −20° C. The sequencing work was completed by Suzhou Genwiz Biotechnology Co., Ltd.

### Example 2 Expression and Purification of GAD Mutant in Escherichia coli BL21

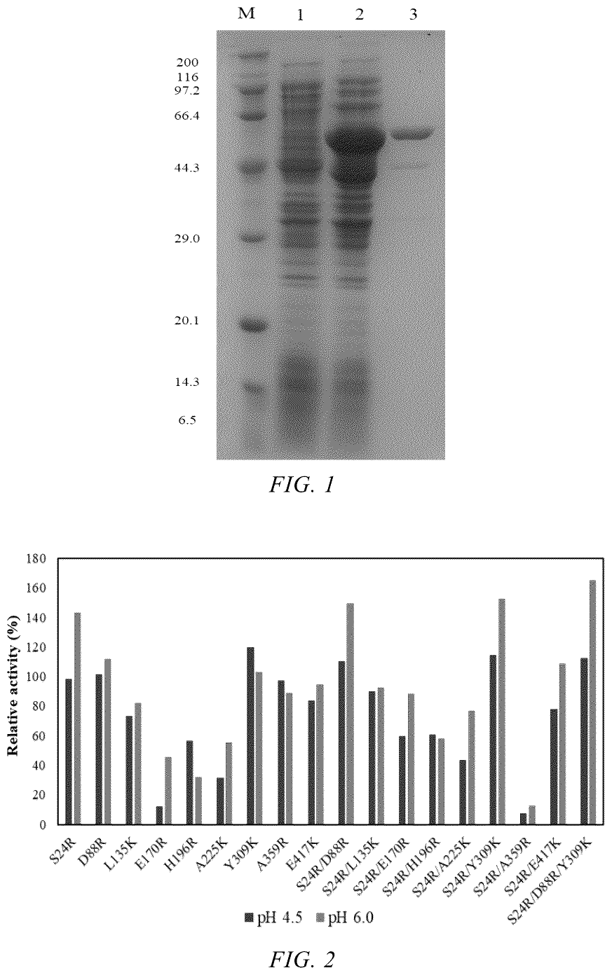

The recombinant plasmid pET-28a containing the mutant gene was transformed into Escherichia coli BL21 by chemical transformation. After the recombinant Escherichia coli was induced at 16° C. for 12 h, homogenized bacterial cells were collected by centrifugation. Protein purification was carried out by nickel column. The purified enzyme was added with 10% glycerol and stored at 4° C. for later use. The purified enzyme was analyzed by SDS-PAGE. The results of E. coli BL21/pET-28a-lpgad^{S24R/D88R/Y309K} are shown in FIG. 1, where M represents protein Marker; lane 1 represents pET28a; lane 2 represents the supernatant of the lpgad^{S24R/D88R/Y309K} mutant; and lane 3 represents the purified lpgad^{S24R/D88R/Y309K} mutant. Results show that a pure recombinant glutamate decarboxylase mutant as determined by agarose electrophoresis was obtained.

### Example 3 Enzyme Activity Assay of Glutamate Decarboxylase and HPLC Analysis of Gamma-Aminobutyric Acid

The relative enzyme activities of a series of purified enzymes (glutamate decarboxylase mutants) obtained in Example 2 when reacting with L-glutamic acid at optimum pH 4.5 and pH 6.0 were determined (where the enzyme activity of the enzyme as shown in SEQ ID NO. 3 was 100%), as shown in FIG. 2. Results show that: the relative enzyme activities of lpgad^{S24R}, l^{pgadS24R/D88R}, lpgad^{S24R/Y309K}, and lpgad^{S24R/D88R/Y309K} were higher, and the relative enzyme activities at pH 6.0 were higher than those at pH 4.5.

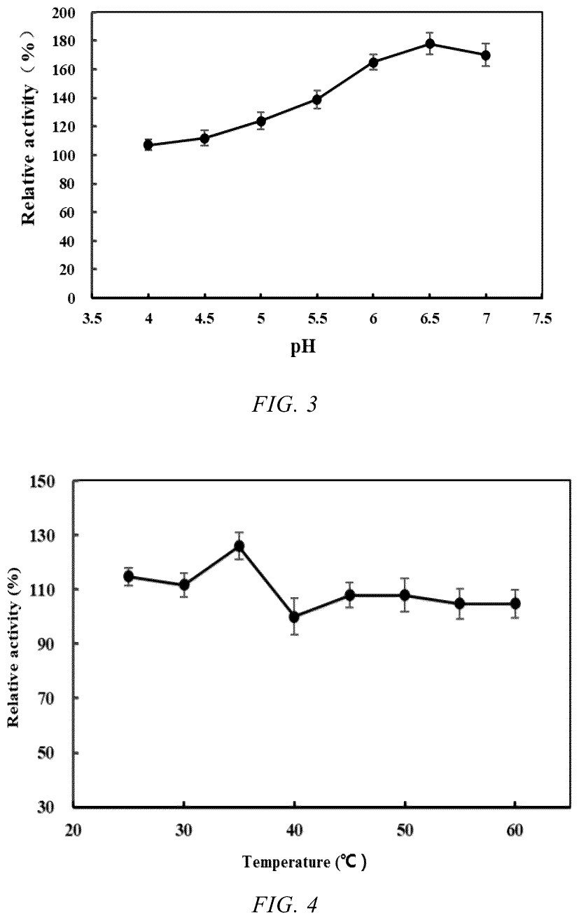

The relative enzyme activities of the glutamate decarboxylase lpgad^{S24R/D88R/Y309K} mutant at pH 4.0, 4.5, 5.0, 5.5, 6.0, 6.5 and 7.0 (as shown in FIG. 3, it can be seen that the relative enzyme activity was the highest at pH 6.5, and the relative enzyme activity was relatively high at pH 5.0-7.0) and at 25° C., 30° C., 35° C., 40° C., 50° C., 55° C. and 60° C. (as shown in FIG. 4) were also determined. After 30 minutes of reaction, samples were taken and the content of the product gamma-aminobutyric acid was determined by HPLC.

Definition of enzyme activity unit: One enzyme activity unit (U) is equal to the amount of enzyme needed to catalyze L-glutamic acid to produce 1 nmol gamma-aminobutyric acid at 30° C. in 1 min. The specific enzyme activity is the enzyme activity of 1 mg wet cell, measured in U/mg. The 1 mL reaction system for decarboxylation reaction between enzyme and substrate adopts the following concentrations: 0.01 mM PLP, and 100 mM L-sodium glutamate. Before the reaction, the buffer solution and enzyme solution were preheated at 30° C. for 5 min, then mixed evenly and reacted at 30° C. for 30 min, finally quickly boiled to terminate the reaction, and centrifuged. The resultant was diluted with 5% trichloroacetic acid (TCA) by a factor of 5. The protein was precipitated in a refrigerator at 4° C. for about 3 h, and then detected by HPLC.

HPLC: The reaction solution was diluted with 5% trichloroacetic acid (TCA) by a factor of 5. The protein was precipitated in a refrigerator at 4° C. for about 3 h, and centrifuged. The supernatant was filtered with a 0.22 jtm membrane, and then injected. Chromatographic column: DinoSoil C18 (5 jtL, 250 mm×4.6 mm), mobile phase: A: 0.1% aqueous formic acid solution; B: 100% acetonitrile; detector: UV Detector; detection wavelength: 360 nm; column temperature: 25° C.; sample size: 10 μL; flow rate: 1.0 mL/min. Process: 0-22 min: 15% B→50% B; 22-22.1 min: 50% B→15% B; 22.1-26 min: 15% B.

### Example 4 Construction of Lpgad^{S24R/D88R/Y309K} in Bacillus subtilis

The pET-28a-lpgad^{S24R/D88R/Y309K} plasmid constructed in Example 1 was used as a template, and was amplified using the primer P1/P20 to obtain a gene of interest. The expression plasmid PMA5 was enzymatically cleaved by the restriction endonuclease BamH I/Mlu I to obtain a linearized vector. A gene of interest was ligated to the linearized vector to construct a recombinant plasmid PMA5-lpgad^{S24R/D88R/Y309K}. The recombinant plasmid was transformed into Escherichia coli JM109 by chemical transformation, coated on an LB plate containing ampicillin, and incubated overnight at 37° C. Clones were randomly picked up, and identified by colony PCR. Results show that the recombinant plasmid containing the gene encoding the glutamate decarboxylase mutant was successfully transformed into the cloning vector Escherichia coli JM109. Colonies determined to be positive were inoculated in a 5 mL LB liquid medium and cultured overnight at 37° C. The recombinant plasmid was extracted from the bacterial solution, and stored in a refrigerator at −20° C.

| | | |
|---|---|---|
| P1 | (SEQ ID NO. 13): | ATGGCAATGTTATACGGTAAACACAAT |
| P20 | (SEQ ID NO. 32): | TCAGTGTGTGAATAGGTATTTCTTAGG |

The successfully constructed recombinant plasmid PMA5-lpgad^{S24R/D88R/Y309K} was transformed into Bacillus subtilis BS168 by chemical transformation to construct a BS168/PMA5-lpgad^{S24R/D88R/Y309K} recombinant strain, which was coated on an LB plate containing kanamycin and cultured overnight at 37° C. Clones were randomly picked up, and identified by colony PCR. The successfully transformed bacterial solution was added with glycerol and stored in a refrigerator at −80° C.

### Example 5 Preparation of Gamma-Aminobutyric Acid from BS168/PMA5-lpgad^{S24R/D88R/Y309K} Engineered Bacterium

The BS168/PMA5-lpgad^{S24R/D88R/Y309K} mutant engineered strain in Example 4 was used for transformation of L-glutamic acid substrate. In a 5 L tank with 1 L 0.9% NaCl solution as the buffer system, the bacterial count OD_{600 nm} was controlled to 16. 100 g/L L-glutamic acid was added in batches, and the reaction was carried out at 30° C. for 12 h. The final yield of 1000 g of substrate fed in batches using the lpgad^{S24R/D88R/Y309K} engineered strain was up to 688.13 g/L, which was about 52% higher than that of the original enzyme, and the molar conversion rate was up to 98.2%. The results show that the catalytic efficiency of the mutant enzyme was significantly improved, providing broad industrial application prospects.

Apparently, the above-described embodiments are merely examples provided for clarity of description, and are not intended to limit the implementations of the invention. Other variations or changes can be made by those skilled in the art based on the above description. The embodiments are not exhaustive herein. Obvious variations or changes derived therefrom also fall within the protection scope of the invention.

What is claimed is:

1. A glutamate decarboxylase mutant with improved pH tolerance, obtained by mutating glutamate decarboxylase having an amino acid sequence as shown in SEQ ID NO. 3 in any of ways selected from:
(1) mutating serine at position 24 to arginine;
(2) mutating serine at position 24 to arginine, and mutating aspartic acid at position 88 to arginine;
(3) mutating serine at position 24 to arginine, and mutating tyrosine at position 309 to lysine; and
(4) mutating serine at position 24 to arginine, mutating aspartic acid at position 88 to arginine, and mutating tyrosine at position 309 to lysine.

2. A gene encoding the glutamate decarboxylase mutant with improved pH tolerance according to claim 1.

3. The gene according to claim 2, wherein a nucleotide sequence of the gene is as shown in SEQ ID NO. 2, SEQ ID NO. 8, SEQ ID NO. 10, or SEQ ID NO. 12.

4. A recombinant expression vector carrying the gene according to claim 2.

5. The recombinant expression vector according to claim 4, wherein pET-28a, PMA5, or PXMJ-19 is used as an original expression vector.

6. A recombinant strain comprising the recombinant expression vector according to claim 4.

7. The recombinant strain according to claim 6, wherein Escherichia Coli, Bacillus subtilis, or Corynebacterium glutamicum is used as a host strain of the recombinant strain.

8. The recombinant strain according to claim 7, wherein the Bacillus subtilis comprises Bacillus subtilis 168.

9. Use of the glutamate decarboxylase mutant with improved pH tolerance according to claim 1.

10. The use according to claim 9, wherein gamma-aminobutyric acid is produced by using L-glutamic acid or L-sodium glutamate as a substrate and the glutamate decarboxylase mutant with improved pH tolerance.
